(19) United States
(12) Patent Application Publication
(10) Pub. No.: US 2025/0248809 A1
(43) Pub. Date: Aug. 7, 2025

(54) Transcatheter Prosthetic Atrioventricular Valve

(71) Applicant: St. Jude Medical, Cardiology Division, Inc., St. Paul, MN (US)

(72) Inventors: Randolf Von Oepen, Aptos, CA (US); William H. Peckels, Robbinsdale, MN (US); Preston James Huddleston, Maplewood, MN (US)

(73) Assignee: St. Jude Medical, Cardiology Division, Inc., St. Paul, MN (US)

(21) Appl. No.: 19/189,455

(22) Filed: Apr. 25, 2025

Related U.S. Application Data
(63) Continuation of application No. PCT/US23/84578, filed on Dec. 18, 2023.
(60) Provisional application No. 63/477,062, filed on Dec. 23, 2022.

(51) Int. Cl. A61F 2/24

(52) U.S. Cl. CPC A61F 2/2418; A61F 2210/0057; A61F 2210/0071; A61F 2220/0033; A61F 2250/0021

(57) ABSTRACT

A prosthetic heart valve for replacing a native atrioventricular valve may include a collapsible and expandable frame including an atrial disk, a ventricular disk, and a center portion extending between the atrial disk and the ventricular disk. A plurality of prosthetic leaflets may be mounted inside the frame, and an outer fabric may be coupled to the frame, the outer fabric extending from the atrial disk to the ventricular disk. One or more members may each have opposite ends coupled to the atrial and ventricular disk, and a center portion that is not coupled to the central portion of the frame. The one or more members may be positioned radially inward of the outer fabric. As the frame expands from a collapsed condition to an expanded condition, the one or more members urge the outer fabric radially outwardly relative to a central longitudinal axis of the frame.

### CROSS-REFERENCE TO RELATED APPLICATIONS

This application is a continuation of International Patent Application No. PCT/US23/84578, filed Dec. 18, 2023, which claims the benefit of the filing date of U.S. Provisional Patent Application No. 63/477,062, filed Dec. 23, 2022, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after expansion (or re-expansion). Using multiple steps and/or multiple delivery system devices may increase the time and complexity of a procedure.

Native atrioventricular valves (i.e., the tricuspid valve and the mitral valve) typically have a larger size and/or diameter compared to the native aortic valve and the native pulmonary valve. Among the native atrioventricular valves, a regurgitant tricuspid valve typically has a larger size and/or diameter than a regurgitant mitral valve. For example, for patients with severe tricuspid valve regurgitation, the diameter of the tricuspid valve may range from about 40 mm to about 66 mm, although these numbers are merely exemplary. As a result, prosthetic heart valve designs and considerations for replacing the different native heart valves are not identical. For example, to accommodate the large size of the mitral and tricuspid valve, recent prosthetic heart valve designs have included an outer frame with a large size to engage the native mitral or tricuspid annulus, and a smaller and generally cylindrical inner frame within that outer frame, the inner frame housing the prosthetic valve leaflets. However, this double-stented design generally increases the bulk of the prosthetic heart valve, resulting in a larger profile when collapsed within a delivery device. This, in turn, requires the delivery device (e.g., a catheter housing the collapsed prosthetic heart valve for delivery) to have a larger size to accommodate the large prosthetic heart valve. Typically, it is desirable for catheters of transcatheter heart valve delivery devices to have a smaller size, since the catheters may need to pass through the vasculature to reach the native heart valve in a minimally invasive manner. Thus, it would be desirable to have a prosthetic heart valve that is able to fit within a native tricuspid or mitral valve but is able to collapse to a small size to be accommodated in a relatively small profile delivery device.

### BRIEF SUMMARY OF THE DISCLOSURE

According to one aspect of the disclosure, a prosthetic heart valve is for replacing a native atrioventricular valve. The prosthetic heart valve may include a collapsible and expandable frame, the frame including an atrial disk, a ventricular disk, and a center portion extending between the atrial disk and the ventricular disk. A plurality of prosthetic leaflets may be mounted inside the frame. An outer fabric may be coupled to the frame, the outer fabric extending from the atrial disk to the ventricular disk. One or more members may each have a first end portion coupled to the atrial disk, a second end portion coupled to the ventricular disk, and a center portion that is not coupled to the central portion of the frame, the one or more members being positioned radially inward of the outer fabric. As the frame expands from a collapsed condition to an expanded condition, the one or more members may urge the outer fabric radially outwardly relative to a central longitudinal axis of the frame. The frame may be formed as a monolithic structure. The frame may be self-expanding. The one or more members may be elastic members. The one or more members may be formed of silicone or thermoplastic polyurethane. In the expanded condition of the frame, the one or more elastic members may be under tension. The one or more elastic members may have a length capable of stretching between 100% and about 500% upon the frame transitioning from the collapsed condition to the expanded condition.

The one or more members may be a plurality of individual elastic members spaced around a circumference of the frame. In the expanded condition of the frame, each of the individual elastic members may extend in a direction substantially parallel to the central longitudinal axis of the frame. In the expanded condition of the frame, each of the individual elastic members may slope radially inwardly in a direction from the atrial disk toward the ventricular disk, or radially inwardly in a direction from the ventricular disk and the atrial disk. The first end portion and the second end portion of each of the individual elastic members may include a ball, the ball having a diameter that is greater than a diameter of the center portion of each of the individual elastic members. The first end portion and the second end portion of each of the individual elastic members may be attached to the frame via a corresponding slot, each slot having a first portion with a diameter that is greater than the diameter of the ball, and a second portion with a width that is smaller than the diameter of the ball.

The one or more members may be an elastic fabric. A low-friction fabric may be positioned on at least one side of the elastic fabric, the low-friction fabric having a lower friction than the elastic fabric. The one or more members may be a braid formed by elastic filaments or by non-elastic filaments. The braid may extend in a braid direction between the atrial disk and the ventricular disk, the braid including warp and weft oriented at an oblique angle relative to the braid direction.

The one or more members may be a non-elastic wire. The non-elastic wire may be a loop having a first horizontal portion at the atrial disk, a second horizontal portion at the ventricular disk, and two vertical portions each coupling the first horizontal portion to the second horizontal portion. The two generally horizontal portions may increase in length as the frame transitions from the collapsed condition to the expanded condition, and the two generally vertical portions may decrease in length as the frame transitions from the collapsed condition to the expanded condition.

### BRIEF DESCRIPTION OF THE DRAWINGS

FIG. 1 is a cross-section of a prosthetic heart valve, taken along section line 1-1 of FIG. 2.

FIG. 2 is a schematic side view of the prosthetic heart valve of FIG. 1.

FIG. 3 is a side view of the prosthetic heart valve of FIGS. 1-2.

FIG. 4 is a top or atrial view of the prosthetic heart valve of FIGS. 1-2.

FIG. 5 is a bottom or ventricular view of the prosthetic heart valve of FIGS. 1-2.

FIG. 6 is a perspective view of the stent of the prosthetic heart valve of FIGS. 1-2 with other components of the prosthetic heart valve omitted.

FIG. 7 is a view of a portion of the cut pattern of the stent of FIG. 6.

FIG. 8A shows the prosthetic heart valve of FIG. 1 with one or more elastic members provided thereon.

FIG. 8B is a schematic top (atrial) view of the prosthetic heart valve of FIG. 8A.

FIG. 8C is a perspective view of the prosthetic heart valve of FIGS. 8A-B.

FIG. 8D is a view of the cut pattern of the stent of FIG. 8C.

FIG. 8E shows the prosthetic heart valve of FIG. 8A implanted in a native valve annulus.

FIG. 8F shows an exemplary loop structure for coupling the elastic members and the frame of FIG. 8A.

Fig, 8G shows an exemplary elastic member structure for use with the loop structure of FIG. 8F.

FIG. 9A shows the prosthetic heart valve of FIG. 1 with one or more elastic members provided thereon in a different position than that shown in FIG. 8A.

FIG. 9B is a view of the cut pattern of the stent of FIG. 9A.

FIG. 10A shows the prosthetic heart valve of FIG. 1 with one or more elastic members provided thereon in a different position than that shown in FIG. 8A.

FIG. 10B is a view of the cut pattern of the stent of FIG. 9A.

FIG. 11A shows the prosthetic heart valve of FIG. 1 with an elastic fabric provided thereon.

FIG. 11B is a view of the cut pattern of the stent of FIG. 11A.

FIG. 12A shows the prosthetic heart valve of FIG. 1 with one or more braided members provided thereon.

FIGS. 13A-B show different views of the prosthetic heart valve of FIG. 1 with one or more non-elastic wires provided thereon.

FIG. 13C shows a portion of the cut pattern of the frame of FIGS. 6-7 with one or more non-elastic wires provided thereon, in a different configuration than shown in FIGS. 13A-B.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure applies with similar force to prosthetic mitral valve replacements. Thus, unless otherwise expressly specified, the embodiments described herein may be used for replacing either a native tricuspid valve or a native mitral valve (with or without additional modifications specific to the heart valve being replaced), even if a particular embodiment may be more suited for replacing either the native tricuspid valve or the native mitral valve.

As explained in the background of the disclosure, prosthetic heart valves that include an anchoring frame and a valve frame nested within the anchoring frame typically have larger sizes when collapsed within a delivery device. As an example, this type of prosthetic heart valve may only fit within a delivery device that has a catheter with an inner diameter that is 30 French (10 mm in diameter) or larger. For transcatheter prosthetic mitral or tricuspid valves that are delivered intravascularly through the femoral vein, a delivery catheter having an outer diameter of 30 French or larger may increase the likelihood of access site complications, which may require a surgeon to intervene. The prosthetic heart valves disclosed herein have features and configurations that are intended to allow for the prosthetic heart valves to have a large enough footprint to reliably anchor within the larger size annulus of the tricuspid valve (or the mitral valve) while being able to collapse into a delivery catheter having an inner diameter of 30 French or smaller. It should be understood that, as used herein, the unit French refers to the inner diameter of a catheter when describing the ability of a valve to fit within that catheter, whereas the unit French refers to the outer diameter of the catheter when describing how catheter size may result in vascular access problems.

As is described below, one way to achieve this functionality is to design the prosthetic heart valve with a single support stent (e.g., a single stent layer or a non-nested frame configuration) that can span the large atrioventricular valve annulus diameters found in patients who experience heart failure. The geometry of the support stent may allow for prosthetic leaflets to be secured inside, with atrial and/or ventricular flanges or disks that have a large enough diameter or profile to clamp the native annulus tissue therebetween. To provide adequate sealing between the support stent and the native valve annulus, fabric(s) may span the gap between the atrial and ventricular disks, where the fabric(s) are capable of elongating to mitigate the effects of foreshortening when sheathing the prosthetic heart valve into the catheter. While the embodiments described below may be suitable for replacing either a tricuspid or mitral valve, these embodiments may be best suited for replacing a tricuspid valve due to the lower right ventricular pressures, compared to left ventricular pressures, which may reduce the need for a nested stent design.

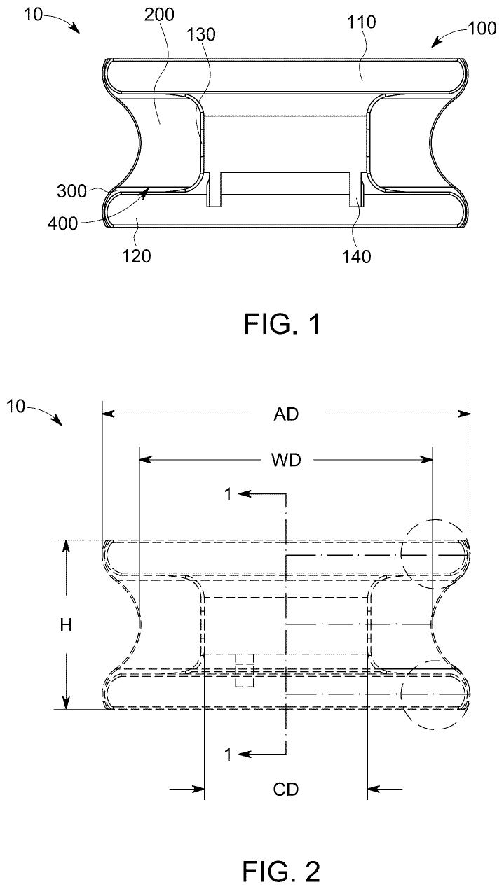

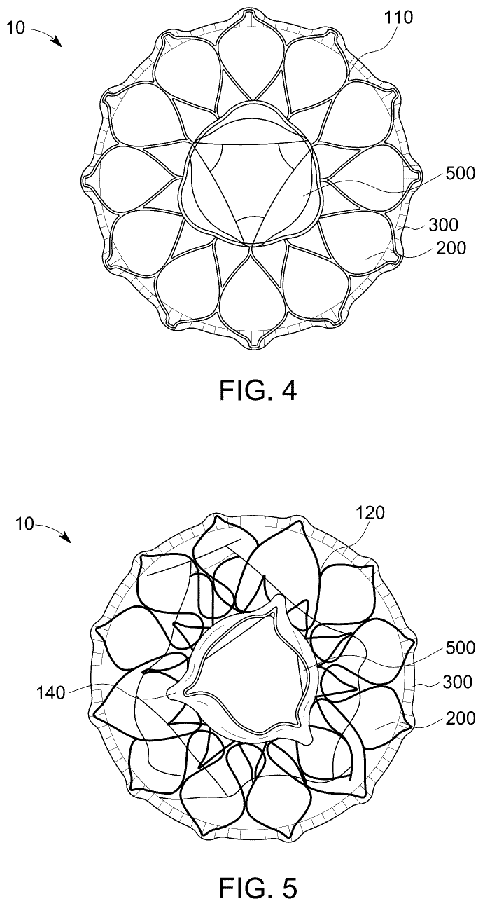

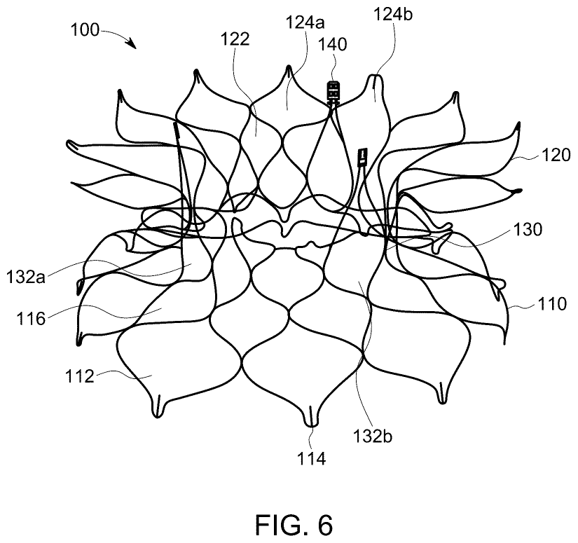

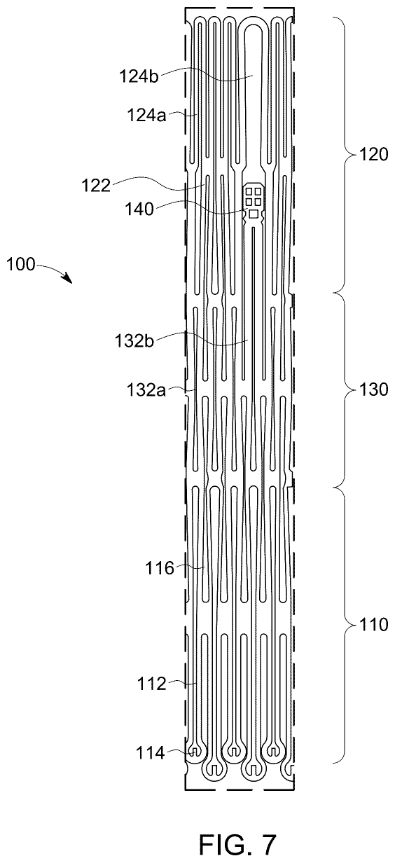

FIG. 1 is a cross-section of a prosthetic heart valve 10, taken along section line 1-1 of FIG. 2, according to one aspect of the disclosure. Generally, prosthetic heart valve 10 includes a support frame or stent 100, which may include an atrial flange 110 (which may alternately be referred to as an atrial disk or anchor), a ventricular flange 120 (which may alternately be referred to as a ventricular disk or anchor), and a central stent portion 130. The stent 100 may also include a plurality of commissure attachment features (“CAFs”) 140 for use in coupling the prosthetic valve leaflets 500 to the stent 100. The prosthetic valve leaflets 500 are omitted from the illustration of FIGS. 1-2 and are best shown in FIGS. 4-5. The stent 100 is described in greater detail below in connection with FIGS. 6-7. Referring still to FIG. 1, the prosthetic heart valve 10 may include a first fabric 200 generally surrounding the central portion 130 of the stent 100 and spanning the gap between the atrial disk 110 and the ventricular disk 120. In one embodiment, the first fabric may be formed as a knitted fabric (e.g., polyethylene terephthalate (“PET”), polytetrafluoroethylene (“PTFE”), ultra-high molecular weight polyethylene (“UHMWPE”), polyester, or similar materials). The formation of the first fabric 200 as a knitted fabric may allow for significant stretching of the first fabric, e.g., up to doubling or tripling in length upon being stretched. In one embodiment, the first fabric 200 is configured to stretch to increase its length by a factor of about 2.5. When in the expanded or deployed condition shown in FIG. 1, a substantial amount of open space or volume may exist between the first fabric 200 and the outside of the stent 100, at least prior to implantation. A second fabric 300 may be provided on the outer surface of the atrial disk 110 and the ventricular disk 120, the second fabric 300 being coupled to the first fabric 200 at seams 400, which may be for example ultrasonic welding seams. In one embodiment, the second fabric 300 may be formed as a woven fabric (e.g., PET, PTFE, UHMWPE, etc.). By forming the second fabric 300 as a woven fabric, the second fabric 300 may be particularly suited to provide sealing functionality against the native anatomy, while the knitted fabric 200 is particularly suited to provide stretching capabilities to allow for relatively unhindered collapsing and expanding of the stent 100. Even if the first fabric 200 is formed as a knit fabric and the second fabric 300 is formed as a woven fabric, it may be desirable (although not required) for the two fabrics to be formed of the same material, with the stretch and/or sealing properties being influenced, at least in part, by the way in which the threads of the fabric are wound, knitted, and/or woven. For example, changing the bias of the warp versus the weft will change the stretch capabilities of a fabric.

Referring briefly to FIG. 2, various exemplary dimensions of the prosthetic heart valve 10 when the prosthetic heart valve 10 is in an expanded or deployed configuration are provided. However, these dimensions are merely exemplary and other dimensions may be suitable. For example, the outer anchor diameter AD of the atrial disk 110 and/or ventricular disk 120 may be about 65 mm. However, in other embodiments, the anchor diameter A D may be between about 50 mm and about 80 mm. In the expanded or deployed condition (e.g., upon implantation into the native tricuspid valve annulus), the first fabric 200 may form a waisted shape with a minimum waist diameter WD near an axial center of the first fabric 200. In the illustrated embodiment, the waist diameter WD may be about 52 mm, but in other embodiments, the waist diameter WD may be between about 45 mm and about 75 mm. The center portion 130 of the stent 100 may be generally cylindrical, and in the illustrated embodiment has a central diameter CD of about 29 mm, but in other embodiments, the central diameter CD may be between about 25 mm and about 35 mm. The prosthetic heart valve 10 may have a height H between the inflow and outflow ends of about 30 mm, but in other embodiments, the height may be between about 25 mm and about 45 mm. As noted above, each of these dimensions is merely illustrative. In some embodiments, in the expanded or deployed condition, the ratio of the outer anchor diameter AD to the central diameter CD is between about 2.5:1 and about 1.5:1, preferably between about 2.5:1 and about 2.0:1, including about 2.25:1. As is explained in greater detail below, this large disparity may allow the atrial disk 110 and ventricular disk 120 to be large enough to provide anchoring, but the central portion 130 to be small enough to house an appropriately sized set of prosthetic valve leaflets 500, while still maintaining a small crimp profile for delivery, e.g. capable of being delivered within, and deployed successfully from, a delivery device catheter having an inner diameter as small as 24 French (8.0 mm) or even as small as 18 French (6.0 mm). In some embodiments, the prosthetic heart valve 10 may be successfully housed within a catheter having an inner diameter of between 22 French (7.33 mm) and 32 French (10.66 mm), including between about 24 French (8.0 mm) and 28 French (9.33 mm).

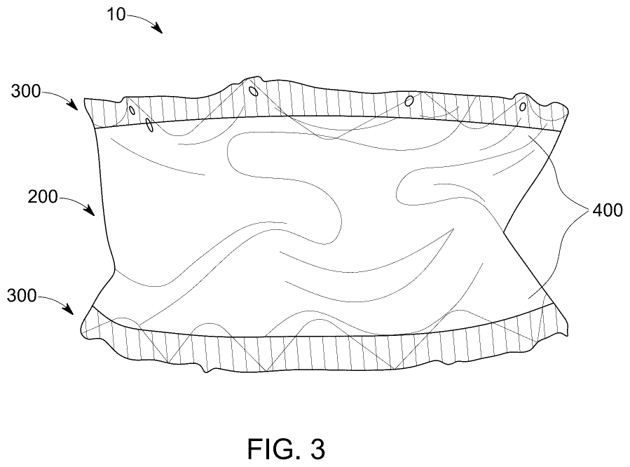

FIG. 3 is a side view of the prosthetic heart valve 10. FIG. 3 illustrates the prosthetic heart valve 10 in an expanded or deployed condition, with the first fabric 200 extending between the atrial disk 110 and the ventricular disk 120. In the illustrated embodiment, first fabric 200 is a stretchy fabric that can easily elongate when the prosthetic heart valve 10 collapses and foreshorten as the prosthetic heart valve 10 expands. To achieve this elongation and foreshortening, it is preferred that the first fabric 200 is not directly coupled to the stent 100. For example, the fabric 200 preferably is not directly sutured to struts of the stent 100. The second fabric 300 includes a portion coupled to the outside of the atrial disk 110 and a portion coupled to the outside of the ventricular disk 110, for example by suturing. As noted above, the second fabric 300 may function to assist with creating or enhancing the seal between the native valve annulus and the atrial and ventricular portions of the prosthetic heart valve 10. The interfaces 400 between the first fabric 200 and the portions of the second fabric 300 are shown in FIG. 3. In the illustrated example, the interfaces 400 are seams created via ultrasonic welding, but the fabrics may be coupled to each other in any suitable way, including suturing, adhesives, etc. In some embodiments, only a single outer fabric may be used, with the single outer fabric having sufficient stretch capabilities to allow the stent 100 to expand and collapse without significant restriction, but while also providing suitable sealing with the native valve annulus.

FIG. 4 illustrates the prosthetic heart valve 10 in an expanded or deployed condition, as viewed from the atrial or inflow side with the prosthetic leaflets 500 in an open condition. FIG. 5 illustrates the prosthetic heart valve 10 in an expanded or deployed condition, as viewed from the ventricular or outflow side with the prosthetic leaflets 500 in an open condition. In the illustrated embodiment, the prosthetic heart valve 10 includes three prosthetic leaflets 500, but in some embodiments, the prosthetic heart valve 10 may include more or fewer prosthetic leaflets. The prosthetic leaflets 500 may be formed of any suitable material. For example, each prosthetic leaflet 500 may be formed of bioprosthetic tissue, such as porcine or bovine pericardium. In other embodiments, each prosthetic leaflet 500 may be formed of a synthetic material such as a synthetic material or fabric, including PET, UHMWPE, PTFE, etc. Each prosthetic leaflet 500 may have two side portions (e.g., forming leaflet tabs), each side portion being coupled to an end of an adjacent prosthetic leaflet 500 to form a commissure, each leaflet commissure being coupled to a respective CA F 140 of the stent 100. Each leaflet may include an outflow edge between the two side edges, the outflow edge being free to allow for opening and closing of the leaflets during normal operation (e.g., to provide the valve functionality). Each leaflet may include an inflow edge between the two side edges, the inflow edge being coupled (e.g., sutured) to the stent 100. In some embodiments, the inflow edges of the prosthetic leaflets 500 are coupled solely (e.g., via sutures) to struts of the central portion 130 of the stent 100, without any direct attachment to other components. In other embodiments, a short fabric skirt may be provided around a portion of the inflow side of the central portion 130 of the stent 100, with the short fabric skirt coupled to the stent 100 and providing an additional structure that may be used for coupling (e.g., via sutures) the inflow ends of the prosthetic leaflets 500 to the prosthetic heart valve 10.

By comparing FIG. 4 to FIG. 5, it can be seen that the atrial disk 110 is “closed,” while the ventricular disk 120 is “open.” In other words, as shown in FIG. 4, the inflow ends of the prosthetic leaflets 500 are directly coupled to the stent 100 (and/or a separate short fabric skirt as noted above) so that there is no open flow pathway between the prosthetic leaflets 500 and the outer fabric skirt(s) (e.g., first fabric 200 and second fabric 300, or the single outer fabric if only a single outer fabric is used) at the atrial disk 110. On the other hand, as shown in FIG. 5, the outflow ends of the prosthetic leaflets 500 have a significant radial spacing from the first fabric 200 and the second fabric 300. With this configuration, after implantation of the prosthetic heart valve 10 into the native tricuspid valve, as the right ventricle contracts, the prosthetic leaflets 500 are forced closed and blood tends to flow in the retrograde direction into the space between the prosthetic leaflets 500 and the first fabric 200. The “closed” configuration at the atrial disk 110 ensures that blood cannot actually pass into the right atrium, but the “open” configuration at the ventricular disk 120 allows that retrograde blood to press the first fabric 200 outwardly and into the native valve annulus to enhance the seal between the prosthetic heart valve 10 and the native tricuspid valve annulus.

FIG. 6 is a perspective view of the stent 100 of the prosthetic heart valve 10 in an expanded condition, with other components of the prosthetic heart valve 10 omitted from the figure. The orientation of stent 100 is opposite that shown in FIGS. 1-3. In other words, the top of the view of FIG. 6 is the outflow end and the bottom of the view of FIG. 6 is the inflow end. Stent 100 is preferably formed of a biocompatible shape memory or superelastic material. One particularly suitable example of this stent material is a nickel-titanium alloy, such as nitinol. However, other materials may be suitable. In one example, stent 100 may be formed by laser cutting a hollow tube of nitinol, and then shape-setting the stent 100 to the desired shape, for example by heat treatment. With this configuration, the stent 100 may take the set shape, such as that shown in FIG. 6, in the absence of applied forces. To deliver the prosthetic heart valve 10, the prosthetic heart valve 10 may be collapsed to a small diameter and positioned within a delivery catheter to be passed intravascularly through the patient into the patient's heart. FIG. 7 shows the cut pattern of a portion of stent 100.

Referring collectively to FIGS. 6-7, the stent 100 may be formed with a plurality of rows of generally diamond-shaped cells. In the illustrated example, the atrial disk 110 includes an inflow row of cells 112, which may include a total of twelve cells. A tine or pin 114 may be formed at the inflow apex of each cell 112, the pin extending a short distance in the outflow direction to a free end. Each pin 114 may be sized and shaped so that a suture loop of the delivery device may slip over the pin 114, keeping the stent 110 connected to the delivery device during delivery and deployment. Upon deployment of the prosthetic heart valve 10, each suture loop may be pushed forward or distally to disengage with the corresponding pins 114 to fully decouple the prosthetic heart valve 10 from the delivery device. Similar pins and suture loops are described in more detail in U.S. Pat. No. 10,874,512, the disclosure of which is hereby incorporated by reference herein.

A transition row of cells 116 may be positioned directly adjacent to the inflow row of cells 112, with cells 116 transitioning between the atrial disk 110 and the central portion 130. In the illustrated embodiment, a total of twelve atrial transition cells 116 are provided. The central portion 130 may be generally formed by a row of central cells, although each central cell may not be identical to each other central cell. In the illustrated example, there are a total of twelve central cells, with a total of nine connected central cells 132a, and three free central cells 132b. Each connected central cell 132a may be diamond-shaped and have both inflow and outflow apices of the central cell 132a connected directly to another cell of the stent. Each free central cell 132b may have an inflow apex coupled directly to the outflow apex of an inflow cell 112, with the outflow apex of the free central cell 132b transitioning into a CAF 140. With this configuration, there are three free central cells 132b evenly spaced around the circumference of the stent 100, and three consecutive connected central cells 132a positioned between each pair of free central cells 132b. In the illustrated embodiment, each CAF 140 is cantilevered in the sense that it is only coupled to the stent 100 on one side, via two struts of the corresponding free central cell 132b. This cantilevered configuration may allow for greater leaflet height while maintaining a relatively small distance between the atrial disk 110 and the ventricular disk 120. The cantilevered configuration may also allow for greater deflection of the CAFs 140 during normal operation of the prosthetic heart valve 10, which may reduce stress on the prosthetic leaflets 500. Each CAF 140 may include one or more eyelets to assist in suturing or otherwise coupling the commissures of the prosthetic leaflets 500 to the CAFs 140. Each CAF 140 may also include a separate fabric coupled to the CAF 140 to assist with suturing of the prosthetic leaflets 500 to the CAF 140. In the illustrated embodiment, each CAF 140 includes a two-by-two array of four small eyelets, with a larger eyelet positioned between the array of four small eyelets and the two struts of the free central cell 132b. However, other configurations of eyelets and other types of CAFs may be suitable as an alternative to CAF 140.

Still referring to FIGS. 6-7, a row of ventricular transition cells 122 may be positioned directly adjacent to the central cells. In the illustrated embodiment, there are a total of six ventricular transition cells 122 provided in pairs, with each ventricular transition cell 122 positioned between two adjacent connected central cells 132. In this example, there are no ventricular transition cells 122 directly adjacent to the free central cells 132b. The ventricular transition cells 122 may form part of the cylindrical central portion 130, and flare outwardly to form part of the ventricular disk 120. In the illustrated embodiment, the final ventricular row of cells includes small ventricular cells 124a and large ventricular cells 124b. For example, a total of nine small ventricular cells 124a may be provided, each with an inflow apex directly connected to the outflow apex of a connected central cell 132a. Between each series (e.g., series of three) small ventricular cells 124a, a large ventricular cell 124b is positioned. Portions of the free central cells 132b, including the CAFs 140, may nest within the large ventricular cell 124b when the stent 100 is collapsed. Thus, in the illustrated embodiment, a total of three large ventricular cells 124b are provided. Although one specific configuration of cells is shown and described in connection with FIGS. 6-7, it should be understood that other configurations of cells may provide for suitable functionality of the prosthetic heart valve 10. With the embodiment described above, the stent 100 may be formed as a single unitary or monolithic structure that, when expanded or deployed, has an hourglass-type shape with two opposite disks having a large diameter for anchoring on the atrial and ventricular sides of the native annulus, with a generally cylindrical center portion extending therebetween having a significantly smaller diameter than the disks. As is explained in greater detail below, the central portion 130 of the stent 100 may be positioned a spaced distance from the native annulus after implantation, which is generally in contrast to typical valves where there is stent structure pushing directly against the native annulus tissue after the prosthetic heart valve is implanted. Rather, as described below, the first fabric 200 directly contacts the native annulus tissue upon implantation, with space remaining between the first fabric 200 and the central portion 130 of the stent 100. By avoiding having two separate stents that overlap and increase the bulk of the prosthetic heart valve when collapsed into a delivery device, prosthetic heart valve 10 may be collapsed into a catheter for delivery, the catheter having an inner diameter of smaller than 30 French, including for example 28 French or 24 French or even smaller. Other benefits of this single stent design may include lower cost, and feasible retrieval of the prosthetic heart valve, as a connector between two nested stents might make retrievability more difficult compared to a single stent design. Although embodiments of prosthetic heart valves described herein are described as having a single frame, it should be understood that, for any of the embodiments described herein, a commissure support or stiffening structure may be included as a component on the prosthetic heart valve. Examples of suitable commissure supports or stiffening structures are described in U.S. Patent Application Publication No. 2024/0164895, the disclosure of which is hereby incorporated by reference.

Referring briefly back to FIGS. 1-3, to allow for the stent 100 to collapse into the delivery device and then expand upon deployment, the outer fabric is preferably designed to not overly restrict the stent 100 from collapsing and expanding. For example, sealing fabrics provided on stents of prosthetic heart valves are frequently woven fabrics and are often tightly sutured or otherwise coupled to the stent so that the fabric does not have the ability to significantly stretch or deviate from its position relative to the stent. Further, these fabrics are often designed with sealing against blood flow as a primary fabric characteristic. On the contrary, prosthetic heart valve 10 has an outer fabric that is capable of significant stretching to accommodate the change in the shape of the stent 100 during transitioning between the collapsed and expanded conditions, with the fabric being spaced a significant distance from the central portion 130 when the prosthetic heart valve 10 is deployed. For example, referring to FIG. 1, when the prosthetic heart valve 10 is deployed into the native valve annulus, the atrial disk 110 and ventricular disk 120 may generally wrap around the atrial and ventricular sides of the native valve annulus, with the outer fabric pressing against the native valve annulus (e.g., via systolic pressure against the fabric on the outflow side), and the central portion 130 of the stent 100 generally centered within (but not directly pressing against) the tissue of the valve annulus. This configuration may allow the central portion 130 of the stent 100 to be a smaller size (e.g., about 28 mm to 32 mm) which may be optimal for hemodynamics, without needing to add additional stent structure to allow for proper sealing and/or anchoring in the much larger sized tricuspid valve annulus. This, in turn, may allow for the prosthetic heart valve 10 to collapse down to a small size for loading into a relatively small delivery device to minimize the likelihood of procedural complications (in particular vascular access complications) resulting from the size of the delivery device. The outer fabric may be formed as a single piece of knit fabric that allows for stretching in one direction, with the ability to seal against the native anatomy and for blood to quickly clot into the fabric to reduce the likelihood of paravalvular (“PV”) leak past the prosthetic heart valve 10 after implantation. However, in other embodiments, such as shown in FIG. 1, the outer fabric may include a first fabric 200 that is formed of a material chosen for its ability to stretch (e.g. a knit fabric), and a second fabric 300 at the atrial disk 110 and ventricular disk 120 that is chosen for the sealing, ingrowth, and/or clotting properties (e.g. a woven fabric), with the first fabric 200 coupled to the second fabric 300 in any desired fashion that allows the stretching to occur.

Although prosthetic heart valve 10 is described above with a particular frame or stent 100, and a particular configuration of sealing fabrics (e.g., first fabric 200 and second fabric 300), it should be understood that these are merely exemplary configurations and other configurations may be suitable. For example, additional configurations of the frame and sealing fabric are described in greater detail in U.S. Patent Application Publication No. 2023/0363904, the disclosure of which is hereby incorporated by reference herein.

As described above, in certain embodiments, the sealing fabric(s) may be “open” on the outflow side of the prosthetic heart valve 10, such that blood may flow in the retrograde direction during ventricular systole into the space between the closed prosthetic leaflets 500 and an interior surface of the sealing fabric(s). This flow will tend to push or balloon or billow the sealing fabric(s) outwardly into contact with the native valve annulus, which may help create a suitable seal against blood flowing around the outside of prosthetic heart valve 10, and/or may help with anchoring of the prosthetic heart valve 10 with the native valve annulus. In some circumstances, the blood pressure that forces the sealing fabric(s) to billow outwardly may be small enough that the outward billowing is not as great as desired to assist with creating the desired sealing and/or anchoring. Thus, in some embodiments, the prosthetic heart valve 10 described above may be modified to include a more “active” feature to assist with pressing the sealing fabric(s) into the native anatomy

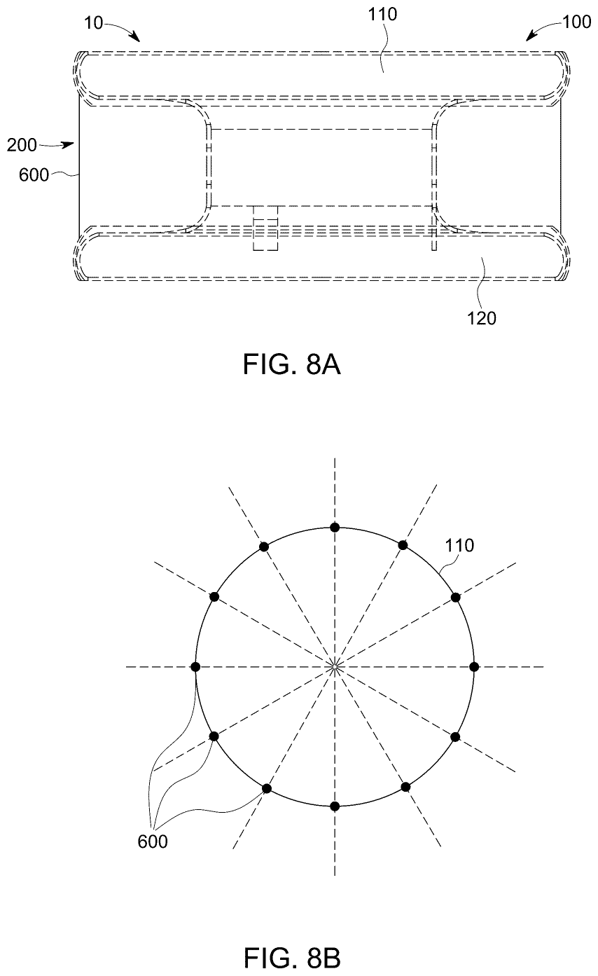

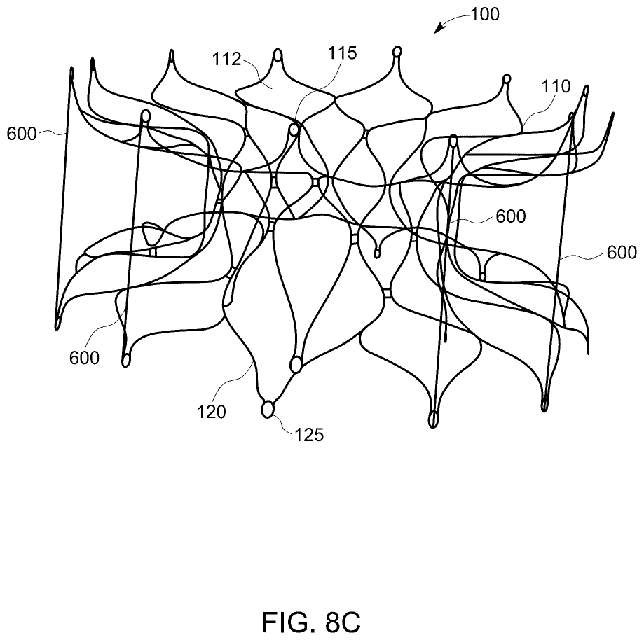

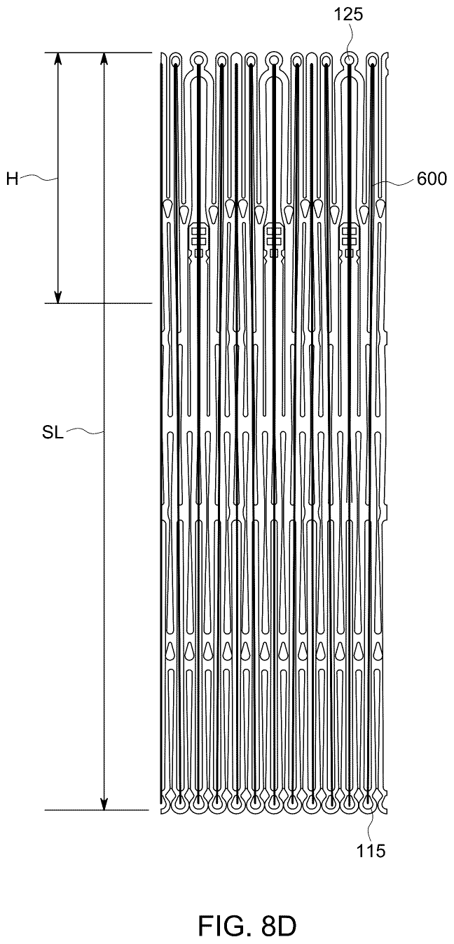

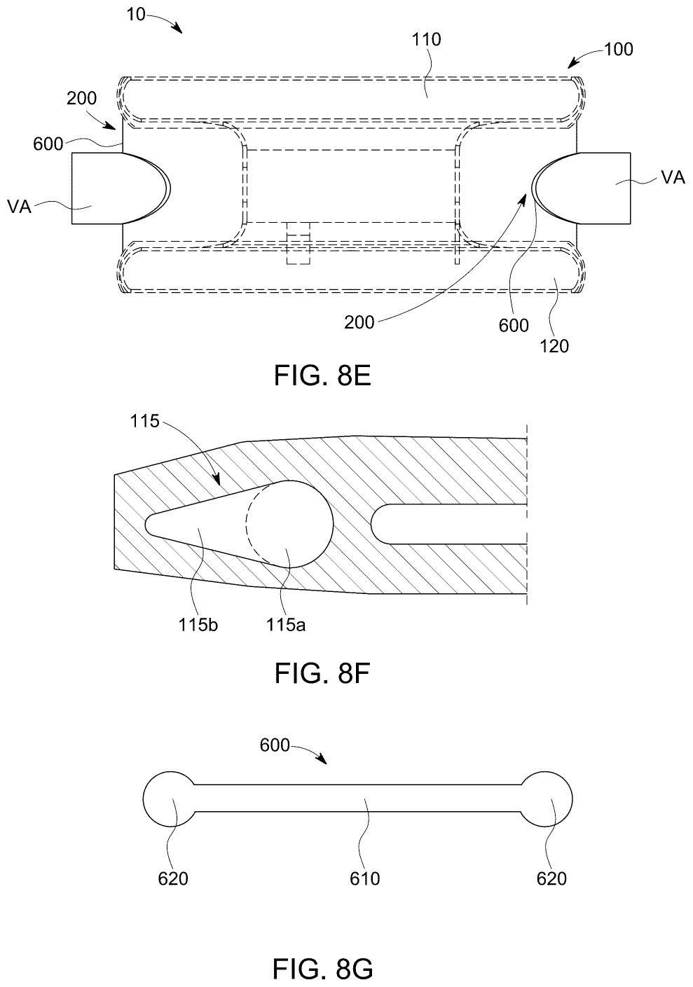

In one example of a mechanism that may help to actively push the sealing fabric(s) into the native valve annulus, one or more elastic members may be added to span from the atrial disk 110 to the ventricular disk 120, with the elastic members tending to push the sealing fabric(s) radially outwardly upon deployment of the frame 100. As shown in FIG. 8A, the prosthetic heart valve 10 may be similar or identical to that shown in FIGS. 1-2, but include one or more elastic members 600, each having one end coupled to the atrial disk 110, and an opposite second end coupled to the ventricular disk 120. The sealing fabric 200 (which may be one-piece or two-pieces similar to that shown in FIG. 1) may be positioned radially outside the elastic members 600. Thus, in FIG. 8A, each elastic member 600 is positioned radially inward of the sealing fabric 200, with the elastic members 600 effectively pressing the sealing fabric 200 outwardly, which is why the sealing fabric 200 and elastic members 600 are not easily distinguishable in FIG. 8A.

The elastic member(s) 600 may be formed of any suitable material that is biocompatible and capable of significant stretching. For example, the elastic member(s) 600 may be formed of silicone rods, thermoplastic polyurethane (“TPU”) rods, elastic fabrics, elastic threads or sutures, or similar materials. Preferably, the elastic member(s) 600 are capable of increasing in length by at least about 100%, at least about 200%, at least about 300%, or more. The elastic member(s) 600 are preferably still in tension when the frame 100 of the prosthetic heart valve 10 is in the expanded or deployed state. With this configuration, the tension on the elastic members(s) 600 tends to actively push the sealing fabric 200 radially outwardly, so that the sealing fabric 200 does not need to rely solely on backpressure to be pressed outwardly into the native valve annulus. The elastic member(s) 600 may be positioned at the farthest radial outward points of the atrial disk 110 and ventricular disk 120 to result in pushing the sealing fabric 200 the maximum distance radially outwardly from the center portion 130 of the frame 100. However, in other embodiments, the elastic member(s) 600 may be positioned at other points on the atrial disk 110 and ventricular disk (e.g., closer to the central portion 130) to fine-tune the distance which the elastic member(s) 600 push the sealing fabric 200 radially outwardly. Regardless of the specific positioning, if no force is applied from the outside, the elastic member(s) 600 will, if under tension, take the shortest path between the two connecting points on the atrial disk 110 and the ventricular disk 120.

If connecting members 600 are used, preferably at least two are used, although more may provide for better functionality. For example, FIG. 8B illustrates a top view (e.g., of atrial disk 110) of the prosthetic heart valve 10 in which a frame 100 includes twelve atrial cells (e.g., atrial cells 112) and twelve ventricular cells (e.g., nine small ventricular cells 124a and three large ventricular cells 124b). With this embodiment, one elastic member 600 may be coupled to the terminal end or tip of each cell, such that a total of twelve elastic members 600 have a first end coupled to the tip of a first atrial cell 112, and a second end coupled to the tip of a correspondingly positioned ventricular cell (e.g., cell 124a or cell 124b) so that each elastic member 600 extends in a direction substantially parallel to the longitudinal axis of the frame 100. In this embodiment, a total of twelve elastic members 600 are provided at substantially equal intervals around the circumference of the prosthetic heart valve 10. However, more or fewer than twelve elastic members 600 may be provided. Similarly, the number of elastic members 600 does not need to directly correspond to the number of cells in the inflow/outflow rows of the frame 100. Further, the elastic members 600 may be spaced at equal intervals, as shown, or unequal intervals, around the circumference of the frame 100.

FIG. 8C is a perspective view of the frame 100 with exemplary positioning of elastic members 600 shown. Although only four elastic members 600 are shown in FIG. 8C, the frame 100 would preferably (but not necessarily) include twelve elastic members 600 if elastic members 600 are provided. The elastic members 600 may be connected to the frame 100 in any suitable fashion, including by knotting, adhesives, laminating, suture connections, or other fasteners, etc. In some embodiments, including that shown in FIG. 8C, the frame 100 may be provided with features to assist in the connection. For example, as shown in FIG. 8C, each atrial cell 112 may include a loop 115 at the tip of the atrial cell 112. Similarly, each ventricular cell (e.g., cell 124a or cell 124b) may include a loop 125 at a tip of the cell. The ends of the elastic members 600 may be coupled to the cells via the loops 115, 125.

FIG. 8D illustrates an exemplary cut pattern of the frame 100, which may be similar or identical to that shown in FIG. 7, but for the loops 115, 125 and the illustration of exemplary positioning of the elastic members 600 between pairs of loops 115, 125. When the frame 100 is collapsed, each elastic member 600 has a stretched length SL that extends substantially the entire axial length of frame 100 when the frame 100 is collapsed. The height H shown in FIG. 8D represents an exemplary height H, relative to the stretched length SL, that the elastic members 600 may have when the prosthetic heart valve 100 is in the expanded or deployed condition. The height H may be longer than an “unstretched length” of the elastic members 600. The “unstretched length” of the elastic members 600 refers to the maximum length of each elastic member 600 in which the elastic members 600 are no longer under tension. Preferably, the height H is larger than the unstretched length so that the elastic members 600 are always under tension, even when the prosthetic heart valve 10 is in the expanded or deployed condition. In some examples, the unstretched length is about 80% of the height H, the unstretched length is preferably any length that is less than 100% of the height H, including about 95% of the height H, about 90% of the height H, about 85% of the height H, etc. In some embodiments, the stretched length SL is about three times the height H, although in other embodiments the stretched length SL may be more or less than three times the height H (e.g., between about 1.5 and 5 times the height H).

FIG. 8E shows the prosthetic heart valve 10 shown in FIG. 8A in a deployed state with representations of portions of the native valve annulus VA. As can be seen in FIG. 8E, the native valve annulus VA pushes against both the sealing fabric 200 and the elastic members 600 positioned radially inward of the sealing fabric 200. Because tension is maintained on the elastic members 600, the elastic members effectively push the sealing fabric 200 into and around the native valve annulus VA to enhance anchoring and/or sealing. This is also because, at least in part, the perimeter of the native valve annulus VA is smaller compared to the perimeter of the sealing fabric 200 and elastic members 600 when the prosthetic valve 10 is in the expanded condition (e.g., sitting on a table with no applied forces). Even if the perimeter of the native valve annulus VA is not circular, the elastic members 600 will help to deform the sealing fabric 200 around, and push the sealing fabric 200 into, the native valve annulus VA. In some embodiments, the active force of the elastic members 600 may be large enough to provide adequate sealing and/or anchoring irrespective of the blood pressure from ventricular systole pushing the sealing fabric 200 outwardly. However, in other embodiments, the active force of the elastic members 600 alone may not be sufficient, but the combination of the active force of the elastic members 600 and the forces from ventricular systole may be enough to provide the desired sealing and/or anchoring of the prosthetic heart valve 10 within the native valve annulus VA. It should be understood that the sealing fabric 200 is preferably not directly connected to the elastic members 600, but the sealing fabric 200 in some embodiments may be connected to the elastic members 600.

Although FIGS. 8A-8E generally show the elastic members 600 as rods or lengths of suture or other elastic members that couple to generally circular loops formed in the frame 100, other configurations are possible. For example, referring back to FIG. 8D briefly, the loops 115 at the tips of the atrial cells may be generally circular, and the loops 125 at the tips of the ventricular cells may be generally circular. However, instead of providing generally circular loops, the loops may have a keyhole shape as shown in FIG. 8F, which may include a generally circular portion 115a that transitions into a necked or generally triangular portion 115b. The same shape may apply to loops 125, as well as additional loops provided within the frame at other locations, as described in greater detail below. The keyhole-shaped loops may be particularly suited for connecting to an elastic member 600 having the configuration shown in FIG. 8G. As shown in FIG. 8G, the elastic member 600 may include a main or center portion 610 having a first width or diameter, and end portions 620 at the ends of the main or center portion 610, the end portions 620 having a generally ball or sphere shape having a second width or diameter. The width or diameter of the end portions 620 is larger than the width or diameter of the center portion 610, for example about twice the width or diameter, although other specific relative sizes may be suitable. The width or diameter of the end portions 620 is about equal to, or slightly smaller than, the diameter of the circular portion 115a of the loop 15, but larger than the width of the narrowed or triangular portion 115b. In use, the end portions 620 of an elastic member 600 may be passed through the circular portions (e.g., 115a) of a loop 115 and a loop 125. The narrowed portions of the two corresponding loops 115, 125 may point toward each other, so the circular portions face toward opposite ends of the frame 100. Because the elastic members 600 are always under tension, the end portions 620 will be drawn toward the narrowed or triangular portions 115b of the loops, and the large size of the end portions 620 relative to the narrowed portions 115b help ensure that the elastic members 600 remain coupled to the frame 100. In this exemplary embodiment, no other mechanism (e.g., adhesives, knots, etc.) are needed to keep the elastic members 600 coupled to the frame 100.

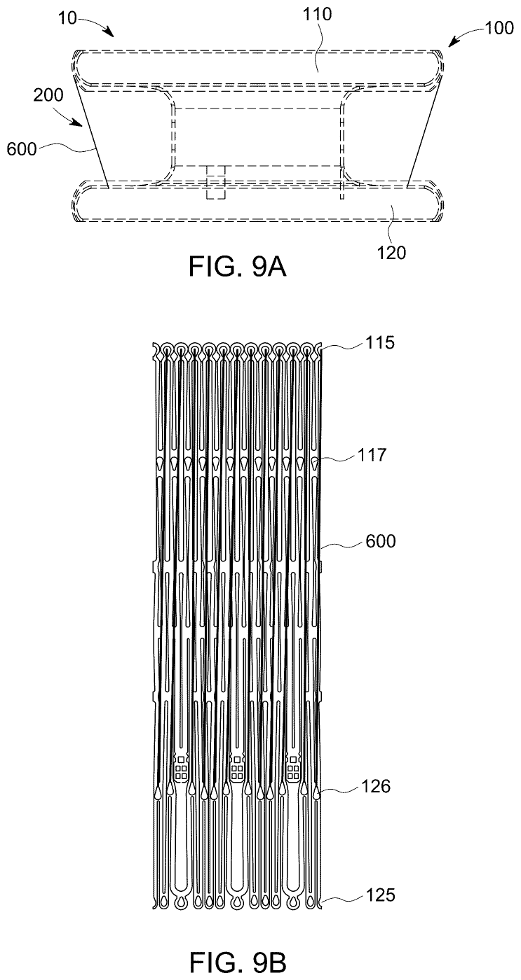

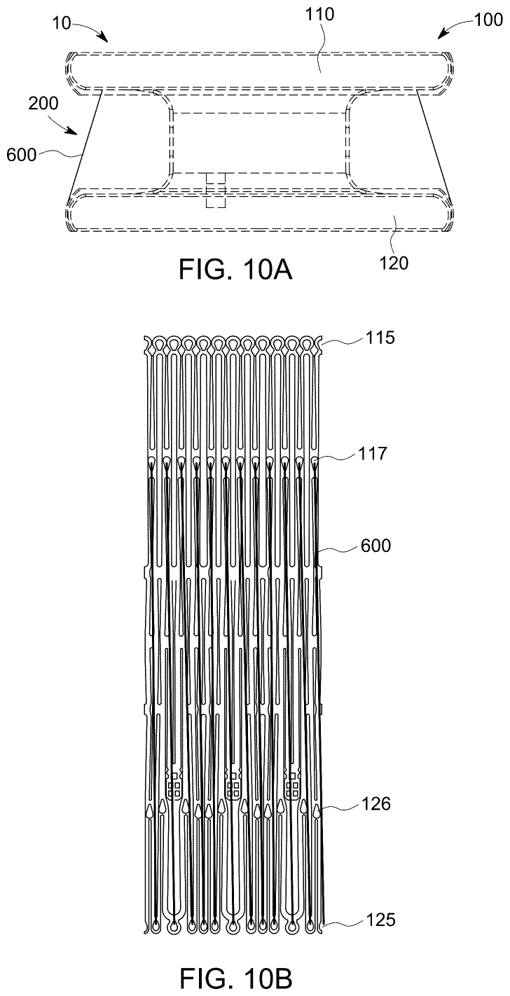

The elastic members 600 of FIG. 8A are shown as having one end attached to a tip of an atrial cell 112, and one end attached to a tip of a ventricular cell (e.g., 124a or 124b). As noted above, this positioning provides for the elastic members 600 to be positioned at or near a maximum outer radial extent of the stent 100. However, other positioning is possible. For example, FIG. 9A shows a prosthetic heart valve 10 with an identical configuration shown in FIG. 8A, except that the ventricular end of the elastic members 600 are coupled to the stent 100 at a different location. In particular, while the atrial ends of the elastic members 600 are still attached to the tips of the atrial cells 112, the ventricular ends of the elastic members 600 are coupled to points where two adjacent ventricular cells meet each other at a ventricular intersection 126. The ventricular intersections 126 may include an opening or loop having the same shape as any of the loops 115, 125 described above, including the shape shown in FIG. 8F. As best shown in FIG. 9A, the result of this positioning is that the elastic members 600 tend to angle inwardly in the atrial-to-ventricular direction. Otherwise, the elastic members 600 shown in FIGS. 9A-B function substantially the same as in the embodiment shown in FIG. 8A, with the elastic members 600 maintaining tension even when the prosthetic heart valve 10 is expanded, resulting in the elastic members 600 helping to push the sealing fabric 200 radially outwardly into the native annulus.

Whereas FIGS. 9A-B show a configuration where the elastic members are angled radially inwardly in the atrial-to-ventricular direction, the opposite is shown in FIGS. 10A-B. In particular, in FIGS. 10A-B, the elastic members have a ventricular end coupled to a tip of the ventricular cells (e.g., loops 125 of cells 124a, 124b), and an atrial end coupled to points where two adjacent atrial cells meet each other at an atrial intersection 117. The atrial intersections 117 may include an opening or loop having the same shape as any of the loops 115, 125 described above, including the shape shown in FIG. 8F. As best shown in FIG. 10A, the result of this positioning is that the elastic members 600 tend to angle inwardly in the ventricular-to-atrial direction. Otherwise, the elastic members 600 shown in FIGS. 10A-B function substantially the same as in the embodiments shown in FIGS. 8A and 9A, with the elastic members 600 maintaining tension even when the prosthetic heart valve 10 is expanded, resulting in the elastic members 600 helping to push the sealing fabric 200 radially outwardly into the native annulus. It should be noted that the orientation of the stent 100 shown in FIGS. 9B and 10B is the opposite of the orientation shown in FIG. 8D.

Although FIGS. 8A, 9A, and 10A illustrate three individual configurations for attaching ends of the elastic members 600 to the stent 100, it should be understood that other options may still be suitable, including for example having both ends of the elastic members attached to points between adjacent cells (e.g., one end attached to intersections 117, the other end attached to intersections 126). And the attachments need not be only to loops or intersections of the stent, and instead the attachments could be directly to struts of the cells. In other words, any suitable attachment points may be used for each end of the elastic members 600 to achieve the desired orientation (e.g., radial positioning, angle) of the elastic members 600 when the prosthetic heart valve 10 is expanded.

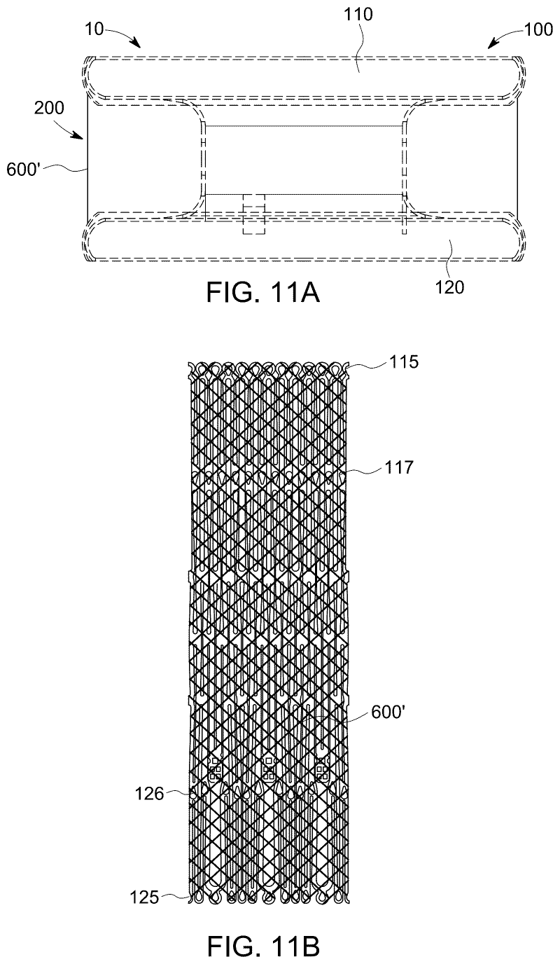

While some of the embodiments shown and described herein include elastic members 600 that are generally in the form of individual tubes, rods, sutures, or threads, other types of elastic members may be used to achieve similar effects. For example, FIGS. 11A-B illustrate an embodiment in which an elastic fabric 600′ is used instead of individual elastic members 600. As shown in FIG. 11B, the elastic fabric 600′a may have an atrial end coupled to the atrial tips of the atrial cells 112 (e.g., via suturing to loops 115) and a ventricular end coupled to the ventricular tips of the ventricular cells (e.g., 124a, 124b via loops 125). If this connection configuration is used, the areas between the atrial and ventricular ends of the elastic fabric 600′ are preferably not directly attached to portions of the stent 100 in between the atrial and ventricular tips. However, as with elastic members 600, the elastic fabric 600′ may have other attachment configurations, including similar to those described in connection with FIG. 9A-B and 10A-B, as well as alternates described therewith. The elastic fabric 600′ may be coupled at the desired coupling locations by any suitable mechanisms, including sutures, adhesives or other fasteners as described in connection with elastic members 600, or by lamination of the elastic fabric 600′ directly to the desired locations on the stent 100.

The elastic fabric 600′ can be formed of TPU, silicone, or other elastic materials, including any of those described in connection with elastic members 600. In one example, the elastic fabric 600′ may be one that can be laminated to the frame, such as the fabric offered by A ran Biomedical under the trademark ValvTEX®. Elastic fabrics may tend to have a relatively high friction and thus be “sticky.” This may generally be undesirable for the contemplated use because the elastic fabric 600′ may increase friction with a delivery device from which the valve must be released, and an increase in friction may lead to an increase in release forces, which may be problematic. In some embodiments, the elastic fabric 600′ may be covered on one or both sides with a thin fabric having low friction to reduce or eliminate the tendency for the elastic fabric 600′ to stick.

The elastic fabric 600′ works in substantially the same fashion as described in connection with the elastic members 600 described above, and thus is not described again here. However, it should be understood that, as with the elastic members 600, the elastic fabric 600′ is preferably not coupled to the sealing fabric 200.

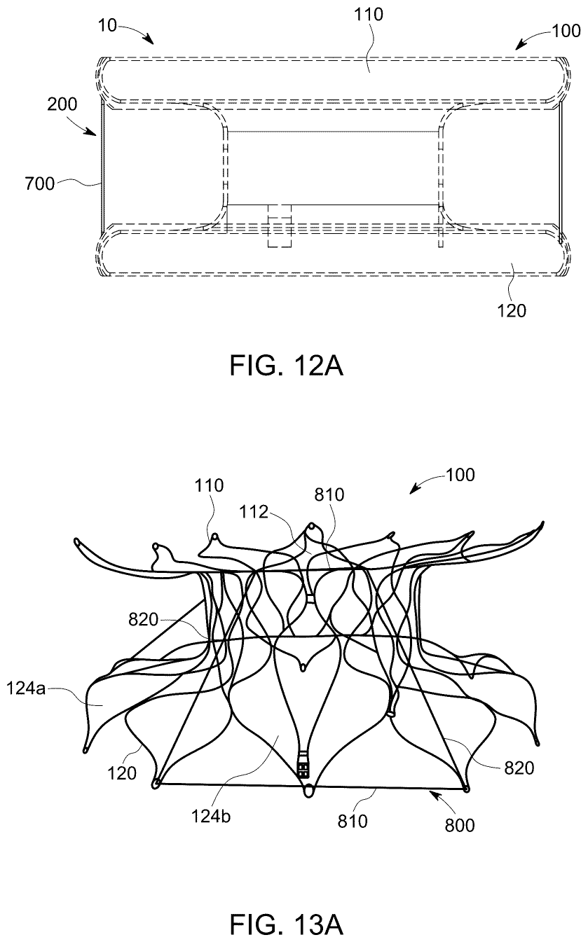

While most of the embodiments above describe the use of one or more elastic members 600 or elastic fabric 600′ that tend to push the sealing fabric 200 outwardly by virtue of the positioning of the members and the tension on the members, other mechanisms may be used instead to achieve a generally similar effect. For example, FIG. 12A shows prosthetic heart valve 10 that is similar or identical to prosthetic heart valve 10 of FIG. 8A, but instead of an elastic member 600 or elastic fabric 600′, a braid 700 may be positioned on the inside of the sealing fabric 200 so that the braid 700 provides outward pressure on the sealing fabric 200 when the prosthetic heart valve 10 is in the expanded condition. The braid 700 may be attached to the frame 100, in a similar or the same way as any of the configurations described above in connection with elastic members 600 (e.g., sutured to the tips of the atrial and ventricular cells). In other embodiments, the braid 700 may be attached to only the sealing fabric 200, or to both the sealing fabric 200 and the frame 100. The warp and weft of the braid 700 is preferably oriented at an oblique angle (e.g., 45 degrees) relative to the inflow-to-outflow direction (e.g., an axis that is parallel to the central longitudinal axis of the prosthetic heart valve 10). When the prosthetic heart valve 10 is transitioned to a collapsed condition, the braid 700 will “stretch” as the prosthetic heart valve 10 elongates axially. On the other hand, when the prosthetic heart valve 10 is transitioned to an expanded condition, the braid 700 will “relax” somewhat, but tension will remain on the braid 700 and cause the braid 700 to push the sealing fabric 200 outwardly into the valve annulus in much the same fashion as described above in connection with elastic members 600 and elastic fabric 600′. The main difference is that the threads or filaments of the braid 700 need not actually be elastic themselves (although they could be), but rather the orientation of the warp and weft allows for the braid 700 to elongate and shorten as the prosthetic heart valve 10 collapses and expands. Although, if the filaments of the braid 700 are indeed elastic, the elastic material may contribute significantly to the ability of the braid 700 to elongate.

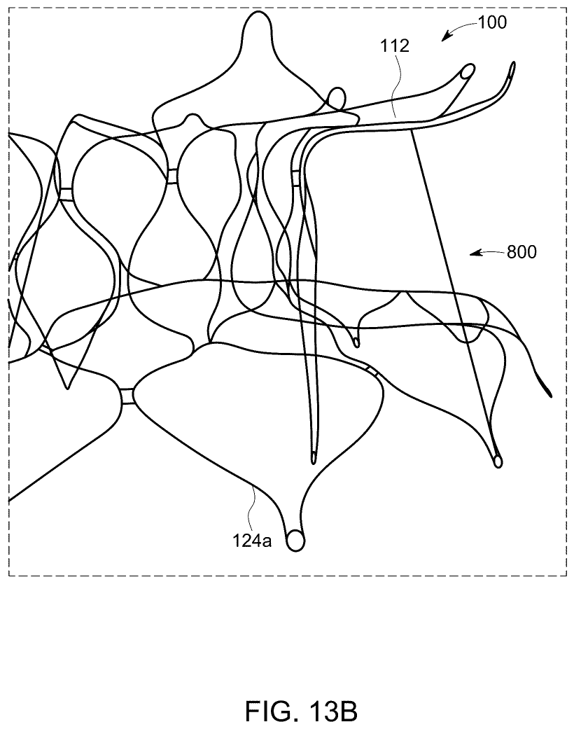

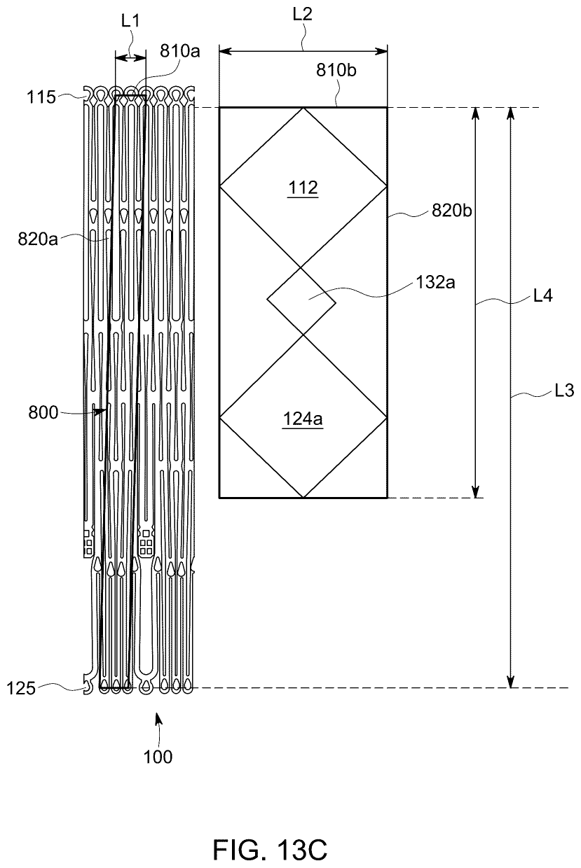

In another embodiment, a non-elastic and/or non-stretchable suture or wire may be used to push the sealing fabric 200 outwardly against the native valve annulus during expansion of the prosthetic heart valve 10. FIG. 13A shows frame 100 of a prosthetic heart valve in an expanded condition. It should be understood that FIG. 13A omits prosthetic leaflets and inner and/or outer skirts from the figure to illustrate features of this embodiment more clearly. It should be understood that, for any of the embodiments described below as having a non-elastic and/or non-stretchable suture or wire, the same design may be used with a stretchable and/or elastic suture or wire.

Referring to FIGS. 13A-B, a non-elastic and/or non-stretchable wire 800 that is coupled to both the atrial disk 110 (e.g., atrial cells 112) and the ventricular disk 120 (e.g., ventricular cells 124a and/or 124b). Because wire 800 is not stretchable or elastic (or at least not appreciably stretchable or elastic), it cannot simply span linearly (e.g., in a direction parallel to the longitudinal axis of the frame 100) between the atrial disk 110 and ventricular disk 120, otherwise the non-elastic nature of the wire 800 would prevent the frame 100 from collapsing. Rather, as shown in FIG. 13A, the wire 800 may be attached to the frame 100 so that it has one or more vertically extending portions and one or more horizontally extending portions that may change length as the frame 100 collapses and expands, while the total length of the wire 800 remains substantially unchanged.

In the particular embodiment illustrated in FIGS. 13A-B, wire 800 extends generally horizontally (or circumferentially) between two adjacent loops 117 in the atrial disk 110, and two loops 125 in the ventricular disk 120 that have one loop 125 between, such that the wire is coupled to tips of small cells 124a on either side of one large cell 124b. In this embodiment, the wire 800 forms a continuous loop with two generally horizontal (or circumferential) extensions 810 that are connected to each other at each end by two generally vertical extensions 820. In the configuration of FIGS. 13A-B, the generally vertical extensions 820 may be “more” vertical when the frame 100 is in the collapsed condition but tend to angle upon expansion of the frame 100 so that the vertical extensions 820 have both vertical and horizontal (or circumferential) extents. In the collapsed condition of the frame 100, the horizontal (or circumferential) extensions 810 may have a relatively small distance, while the vertical extensions 820 may have a relatively large distance. As the frame 100 expands, the horizontal extensions 810 may increase in length, while the vertical extensions 820 may decrease in length. At all times, however, the sum of the distance of the horizontal (or circumferential) extensions 810 and the vertical extensions 820 remains substantially unchanged. With this particular configuration, as shown in FIG. 13B, in the expanded condition of the frame 100, the wire 800 is sloped radially outwardly in the atrial-to-ventricular direction generally similar to that shown in FIGS. 10A-B. However, other variations may be suitable, including a more vertical extension (e.g., more similar to that shown in FIGS. 8A-8G) or a slope radially inwardly in the atrial-to-ventricular direction (e.g., more similar to that shown in FIGS. 9A-B). In all embodiments, when the frame 100 is in the expanded condition, the wire 800 is kept generally taut, so that a sealing fabric 200 positioned radially outward of the wire 800 is pushed outwardly by the wire 800.

The features described above in connection with wire 800 and frame 100 are illustrated in further detail in FIG. 13C, which shows wire 800 coupled the frame 100 in a slightly different configuration than shown in FIGS. 13A-B. In particular, wire 800 in FIG. 13C is coupled to three consecutive loops 115 on atrial disk 110 and three consecutive loops 125 on ventricular disk 120. With this configuration, when the frame is in the collapsed condition, the wire 800 includes two generally horizontal extensions 810a each having a length of about L1, and two generally vertical extensions 820a each having a length of about L3. After the frame 100 expands, represented by the right side of the view of FIG. 13C, the wire 800 has two horizontal extensions 810b each having a length of about L2, and two generally vertical extensions 820b each having a length of about L4. The lengths L1 are smaller than the lengths L2, and the lengths L3 are larger than the lengths L4. The sum of the lengths L1 and L3 are substantially equal to the sum of the lengths L2 and L4. It should be understood that the illustration in FIG. 13C is shown as a two-dimensional object, but the concepts apply generally to three-dimensional objects. It should be understood that, as with the other embodiments described herein, the prosthetic heart valve that incorporates the frame 100 and wires 800 of FIGS. 13A-C include a sealing fabric 200 radially outwards, but preferably not directly connected to, the wire 800.

Although not illustrated, one or more hooks may be added to the sealing fabric 200 of any of the embodiments described above. For the embodiments described above which include an elastic fabric (e.g., fabric 600′) or a braid in/on/adjacent to the sealing fabric 200 (e.g., braid 700), one or more hooks may be coupled to the elastic fabric or braid. The hooks may have one or more pointed or otherwise sharp tips that are configured to engage with native tissue (e.g., the native valve annulus and/or native leaflets) upon expansion of the prosthetic heart valve into the native valve annulus. The hooks may take any suitable shape or configuration, including that described in connection with U.S. Patent Application Publication No. 2022/0280166 and/or U.S. Patent Application Publication No. 2017/0119400, the disclosures of which are hereby incorporated by reference herein.

In an exemplary use of the prosthetic heart valves described herein, a prosthetic heart valve may begin in the expanded condition prior to implantation into a patient. As described above, the prosthetic heart valve may include a single monolithic or unitary stent with an outer fabric on the stent, and any of the features described herein (e.g., elastic members, elastic fabrics, braids or wires) in addition to the outer fabric. The prosthetic heart valve may be drawn or otherwise forced into a delivery catheter, the prosthetic heart valve transitioning into the collapsed condition as it moves into the delivery catheter. Preferably, the outer diameter of the catheter of the delivery device has a size of 36 French (12 mm) or smaller, including 33 French (11 mm) or smaller, including 30 French (10 mm) or smaller, including 28 French (9.33 mm) or 24 French (8 mm). As the prosthetic heart valve is drawn into the catheter of the delivery device, the elastic members or elastic fabrics will stretch if used, while the braid or wire will change configuration if used. With the prosthetic heart valve successfully collapsed in the small diameter delivery device catheter, the delivery device may be introduced into the patient, for example through the femoral vein, and navigated to the target site, for example the native tricuspid valve. Upon reaching the target site, the prosthetic heart valve may be deployed from the delivery device catheter, for example by retracting a capsule or sheath housing the prosthetic heart valve relative to the prosthetic heart valve. For a self-expanding prosthesis, as the constraint on the prosthetic heart valve is removed, the prosthetic heart valve will naturally begin to expand as the stent tends to return to its preset shape. If the prosthesis is balloon-expandable, a balloon may be inflated within the prosthesis to force the valve to expand. Preferably, the ventricular disk of the prosthetic heart valve is released first within the ventricle (e.g., the right ventricle). As the ventricular disk expands, the ventricular disk may create an anchor point on the ventricular side of the native annulus. As deployment continues, the center portion of the stent of the prosthetic heart valve will remain generally centered within the native valve annulus, with little or no direct contact with the native valve annulus. As the atrial side of the prosthetic heart valve deploys, the atrial disk of the stent will expand and create another anchor point on the atrial side of the native valve annulus. During the expansion, the elastic members of elastic fabrics, if used, will shorten (while still maintaining at least some baseline tension), causing the outer sealing fabric to press into and/or conform to the native valve annulus. If the braids or wires are used, they will change configuration to similarly cause the outer fabric to press into and/or conform to the native valve annulus. If additional anchoring features are included, such as sharp-tipped ventricular cells or the hooks described above, those features may tend to dig into or otherwise enhance the anchoring upon expansion of the prosthetic heart valve. Thus, as described above, a small delivery device may be used, despite the requirement of coverage of a large native valve annulus area, and without losing any sealing capabilities despite using only a single stent with a small center portion housing the prosthetic leaflets.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve for replacing a native atrioventricular valve, the prosthetic heart valve comprising:
a collapsible and expandable frame, the frame including an atrial disk, a ventricular disk, and a center portion extending between the atrial disk and the ventricular disk;
a plurality of prosthetic leaflets mounted inside the frame;
an outer fabric coupled to the frame, the outer fabric extending from the atrial disk to the ventricular disk; and
one or more members each having a first end portion coupled to the atrial disk, a second end portion coupled to the ventricular disk, and a center portion that is not coupled to the central portion of the frame, the one or more members being positioned radially inward of the outer fabric,
wherein as the frame expands from a collapsed condition to an expanded condition, the one or more members urge the outer fabric radially outwardly relative to a central longitudinal axis of the frame.

2. The prosthetic heart valve of claim 1, wherein the frame is formed as a monolithic structure.

3. The prosthetic heart valve of claim 2, wherein the frame is self-expanding.

4. The prosthetic heart valve of claim 1, wherein the one or more members are elastic members.

5. The prosthetic heart valve of claim 4, wherein the one or more members are formed of silicone or thermoplastic polyurethane.

6. The prosthetic heart valve of claim 4, wherein in the expanded condition of the frame, the one or more elastic members are under tension.

7. The prosthetic heart valve of claim 6, wherein the one or more elastic members has a length capable of stretching between 100% and about 500% upon the frame transitioning from the collapsed condition to the expanded condition.

8. The prosthetic heart valve of claim 6, wherein the one or more members is a plurality of individual elastic members spaced around a circumference of the frame.

9. The prosthetic heart valve of claim 8, wherein in the expanded condition of the frame, each of the individual elastic members extends in a direction substantially parallel to the central longitudinal axis of the frame.

10. The prosthetic heart valve of claim 8, wherein in the expanded condition of the frame, each of the individual elastic members slopes radially inwardly in a direction from the atrial disk toward the ventricular disk.

11. The prosthetic heart valve of claim 8, wherein in the expanded condition of the frame, each of the individual elastic members slopes radially inwardly in a direction from the ventricular disk and the atrial disk.

12. The prosthetic heart valve of claim 8, wherein the first end portion and the second end portion of each of the individual elastic members includes a ball, the ball having a diameter that is greater than a diameter of the center portion of each of the individual elastic members.

13. The prosthetic heart valve of claim 12, wherein the first end portion and the second end portion of each of the individual elastic members is attached to the frame via a corresponding slot, each slot having a first portion with a diameter that is greater than the diameter of the ball, and a second portion with a width that is smaller than the diameter of the ball.

14. The prosthetic heart valve of claim 6, wherein the one or more members is an elastic fabric.

15. The prosthetic heart valve of claim 14, wherein a low-friction fabric is positioned on at least one side of the elastic fabric, the low-friction fabric having a lower friction than the elastic fabric.

16. The prosthetic heart valve of claim 1, wherein the one or more members is a braid formed by non-elastic filaments.

17. The prosthetic heart valve of claim 16, wherein the braid extends in a braid direction between the atrial disk and the ventricular disk, the braid including warp and weft oriented at an oblique angle relative to the braid direction.

18. The prosthetic heart valve of claim 1, wherein the one or more members is a non-elastic wire or suture.

19. The prosthetic heart valve of claim 18, wherein the non-elastic wire or suture is a loop having a first horizontal portion at the atrial disk, a second horizontal portion at the ventricular disk, and two vertical portions each coupling the first horizontal portion to the second horizontal portion.

20. The prosthetic heart valve of claim 19, wherein the two generally horizontal portions increase in length as the frame transitions from the collapsed condition to the expanded condition, and the two generally vertical portions decrease in length as the frame transitions from the collapsed condition to the expanded condition.
